(19) United States
(12) Patent Application Publication
(10) Pub. No.: US 2022/0152242 A1
(43) Pub. Date: May 19, 2022

(54) System and Method of Sanitizing a Fabric-Based Currency

(71) Applicant: John Stephen McCook, Newnan, GA (US)

(72) Inventor: John Stephen McCook, Newnan, GA (US)

(21) Appl. No.: 17/528,174

(22) Filed: Nov. 16, 2021

Related U.S. Application Data
(60) Provisional application No. 63/205,060, filed on Nov. 16, 2020.
(60) Provisional application No. 63/207,668, filed on Mar. 16, 2021.
(60) Provisional application No. 63/207,669, filed on Mar. 16, 2021.

(51) Int. Cl. A61L 2/10; A61L 2/26; A61L 2/04; A61L 2/20; A61L 2/025

(52) U.S. Cl. CPC A61L 2/10; A61L 2/26; A61L 2/04; A61L 2/202; A61L 2202/11; A61L 2202/26; A61L 2202/17; A61L 2202/122; A61L 2/025

(57) ABSTRACT

A method of sanitizing fabric-based currency eliminates or significantly reduces pathogens on fabric-based currency. The method is implemented by a system that includes an airtight container including a treatment chamber, a chamber entrance/exit, at least one UV lamp, and an ozone dispensing system. The method begins placing at least one piece of fabric-based currency through the chamber entrance/exit and into the treatment chamber. The piece of fabric-based currency is hermetically sealed within the treatment chamber by closing the chamber entrance/exit. The piece of fabric-based currency is then exposed to at least one quantity of UV radiation for a specified period of time. The piece of fabric-based currency is exposed to a quantity of ozone for another specified period of time by pumping the quantity of ozone into the treatment chamber. The chamber entrance/exit is finally opened, and the piece of fabric-based currency is removed from the treatment chamber.

The current application claims a priority to the U.S. Provisional Patent application Ser. No. 63/205,060 filed on Nov. 16, 2020, a priority to the U.S. Provisional Patent application Ser. No. 63/207,668 filed on Mar. 16, 2021, and a priority to the U.S. Provisional Patent application Ser. No. 63/207,669 filed on Mar. 16, 2021.

### FIELD OF THE INVENTION

The present invention generally relates to sanitation processes. More specifically, the present invention is a method of sanitizing a fabric-based currency.

### BACKGROUND OF THE INVENTION

In recent years, there has been an increase in people becoming sick from bacterial and viral infections. Also, over the years, antibiotic resistant pathogens (bacteria and viruses) have been causing more and more illness and deaths. There is even an issue with thermal resistant pathogens on the rise, and that's even more disconcerting. When SARS CoV-2 (Coronavirus, Covid-19) came to the forefront of the world stage, there was a rush to find a cause, then a way to slow and/or stop its spread, and then of course there was a massive onslaught to find a cure or vaccine. The world was in a panic, people were getting sick each day, and each day the numbers grew more and more. Then the virus began to mutate, subtle at first, minor mutations that did not immediately result in higher mortality rates but did increase the infectivity of the virus. Eventually, there was a massive mutation with seventeen variances and that was a signal to a lot of scientists and researchers that this was not going away anytime soon.

Most people know that pathogens are spread through social contacts and surfaces. However, there have been many studies over the years regarding the pathogenic infectivity of money as it moves through society. While the very nature of money is one that cannot be accurately monitored to have even a remotely accurate finding, there have been studies as to the pathogens (bacteria, viruses, and parasites) found on money throughout the world. Money has always been the “unknown vector” when it comes to the spread of disease and this was reflected in the SARS CoV-2 (Covid-19) outbreak near the beginning of 2020 that carried over into 2021 and has been responsible for millions of deaths. While we know that money is a vector, we also have no way to truly and accurately quantify the numbers of infections through something handled by billions of people each and every day. There has been a push to move to a touchless method of payment, running simultaneously with a push to adopt cryptocurrency as a means of payment. However, almost all countries in the world use tangible currency in their everyday lives and this is not something soon to change.

The present invention utilizes multiple approaches to best sanitize currency, specifically fabric-based bills. Further embodiments of the present invention may sanitize metal coins as well. This process provides overlapping sanitizing properties that have proven results and can significantly reduce the total number of pathogenic colonies on one of the most handled items in the world each day. Through colony reduction, there is proof of pathogenic infectivity without the fear of the “bubble boy” theory. The “bubble boy” theory involves the prospect where millions of people want a completely sanitary, thoroughly disinfected environment. This, by its very nature, could result in the deterioration and weakening of the human immune system to the point that the simplest of viruses could overwhelm and kill a healthy person. There are other considerations along this line of thinking as well. The utilization of “far UV-C” has the potential to cleanse an area in a person's home without the implications associated with standard UV-C of causing physical harm to the skin and eyes of the resident(s). However, the ill-conceived notion that most are not considering is that the human body is a host to millions upon millions of beneficial and helpful bacteria. These bacteria keep a human being healthy and in a homeostasis with their surroundings. To remove these bacteria would result in a very certain, and very painful death.

The global market is looking for a solution to this problem, and chemical cleaning agents are not always the solution. The environment used for handling of money is not always conducive to chemical agents and the method involved in sanitizing paper and metal currency. This is where anon-chemical, residue free, historically proven through science method is needed. The short answer to all of the issues going on in the world during 2020, and into 2021, is that there has been an ever-increasing number of viruses, bacteria, and other parasites that are evolving out of science and medicine's wealth of knowledge. Also, there is the ever-increasing concern over antibiotic resistant pathogens. The present invention uses a multi-layered, non-chemical approach that renders most pathogens incapable of reproduction; therefore, reducing the spread of antibiotic resistant pathogens. The non-chemical approach is safer for humans, animals, and the environment.

### BRIEF DESCRIPTION OF THE DRAWINGS

FIG. 1 is a schematic diagram illustrating a system that is used to implement a method of the present invention for a piece of fabric-based currency.

FIG. 2 is a schematic diagram illustrating a system that is used to implement a method of the present invention for a piece of metal-based currency.

FIG. 3 is a flowchart illustrating the overall process for a method of the present invention.

FIG. 4 is a flowchart illustrating the subprocess of exposing the piece of fabric-based currency to a quantity of type-C UV radiation, to a quantity of type-B UV radiation, and then to a quantity of type-A UV lamp.

FIG. 5 is a flowchart illustrating the subprocess of exposing the piece of fabric-based currency to another quantity of UV radiation after being exposed to a quantity of ozone.

FIG. 6 is a flowchart illustrating the subprocess of heating the piece of fabric-based currency before being exposed to the quantity of ozone.

FIG. 7 is a flowchart illustrating the subprocess of heating the piece of fabric-based currency after being exposed to the quantity of ozone.

FIG. 8 is a flowchart illustrating the subprocess of washing and vibrating a piece of metal-based currency.

FIG. 9 is a flowchart illustrating the subprocess of drying the piece of metal-based currency after being washed and vibrated.

### DETAIL DESCRIPTIONS OF THE INVENTION

All illustrations of the drawings are for the purpose of describing selected versions of the present invention and are not intended to limit the scope of the present invention.

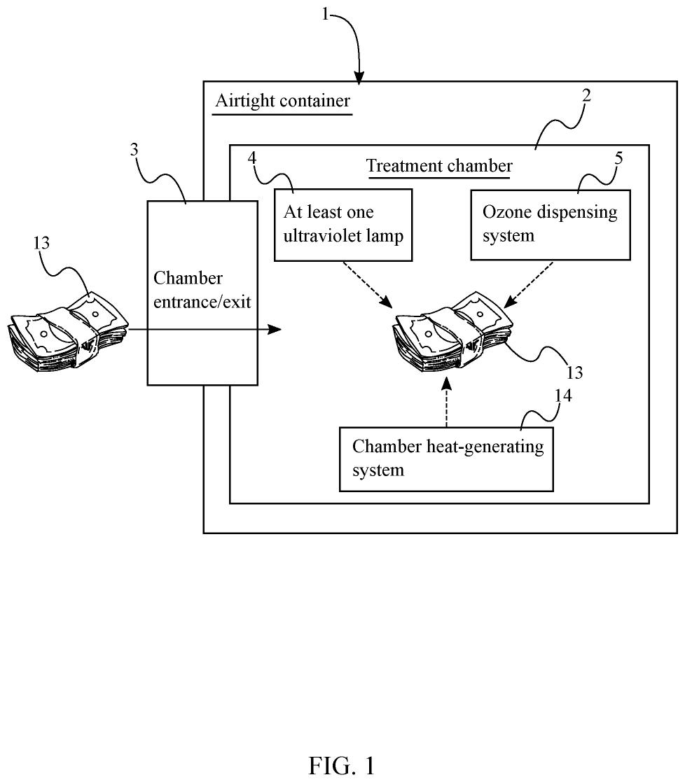

The present invention is a system and method of sanitizing a fabric-based currency. The present invention ensures safer, sanitary materials that are handled by individuals at multiple times throughout a day and every day. The present invention is preferably utilized in retail environments and banks as the exchange of physical currency is used for many if not most transactions. The present invention renders harmful viruses, bacteria, and parasites incapable of reproduction or renders viruses, bacteria, and parasites inert, completely. Moreover, the present invention damages, kills, or renders pathogens incapable of reproduction that are known to be harmful and potentially fatal for individuals. The present invention sanitizes materials but does not sterilize or completely disinfect materials. Consequently, the overall number of deaths, severe illnesses, and burden on national medical infrastructure for countries is reduced with the present invention. Thus, the physical system used to implement the method for the present invention includes an airtight container 1, wherein the airtight container 1 includes a treatment chamber 2, a chamber entrance/exit 3, at least one ultraviolet (UV) lamp 4, and an ozone dispensing system 5 (Step A), seen in FIG. 1. The airtight container 1 keeps individuals safe from direct UV lamp 4 exposure and ozone exposure within the treatment chamber 2. In the preferred embodiment of the present invention, the airtight container 1 is portable and secure as the airtight container 1 includes stainless steel walls and mounted onto a chassis. The airtight container 1 preferably includes a layer of reflective material that lines the treatment chamber 2 in order to amplify the UV lamp 4 exposure within the treatment chamber 2. The treatment chamber 2 is the internal compartment that exposes materials to UV lamp 4 and ozone. Materials may be placed within and removed from the treatment chamber 2 through the chamber entrance/exit 3. The at least one UV lamp 4 emits UV lamp 4 of varying wavelengths that target a variety of pathogens. Likewise, the ozone dispensing system 5 releases ozone, preferably O₃, into the treatment chamber 2 in order to cause significant damage to pathogens that the UV lamp 4 is not able to target or reach. It is understood that a supply of ozone is safely stored and connected with the treatment chamber 2 such that individuals are not exposed to any ozone released by the ozone dispensing system 5.

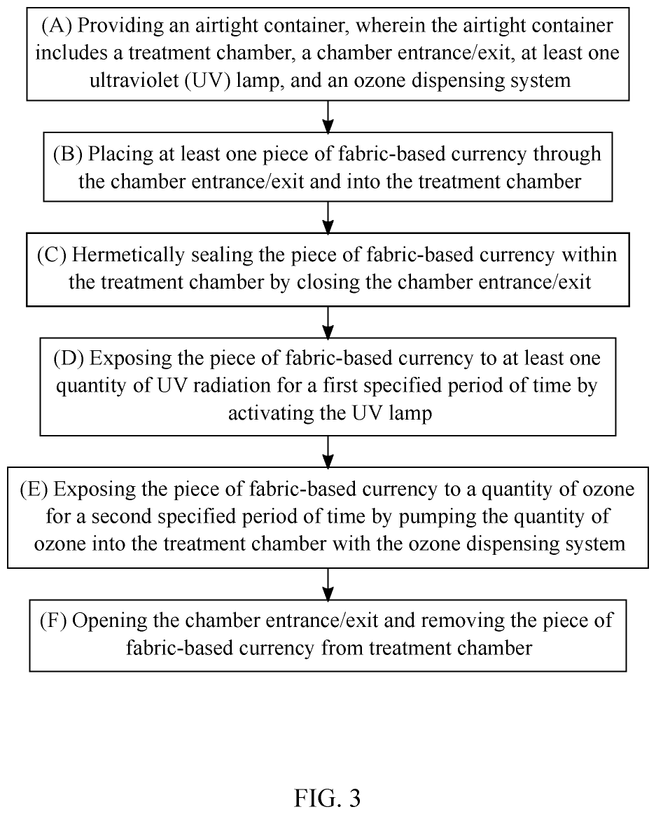

The overall process for the method of the present invention includes the following steps that are implemented with the airtight container 1, the treatment chamber 2, the chamber entrance/exit 3, the at least one UV lamp 4, and the ozone dispensing system 5. As seen in FIG. 3, the overall process begins by placing at least one piece of fabric-based currency 13 through the chamber entrance/exit 3 and into the treatment chamber 2 (Step B), thereby positioning the at least one piece of fabric-based currency 13 within reach of UV lamp 4 exposure and ozone exposure. The at least one piece of fabric-based currency 13 is preferably at least one bill that has been handled during a transaction within a corresponding retail store or bank. In the preferred embodiment of the present invention, the piece of fabric-based currency 13 is made of a quantity of cotton and a quantity of linen as many, if not most, fabric-based currency 13 is made of cotton and linen. More specifically, the quantity of cotton is 75 percent by weight of the piece of fabric-based currency 13, and the quantity of linen is 25 percent by weight of the piece of fabric-based currency 13, as seen in the composition of the American dollar bill. In order to protect individual around the airtight chamber, and specifically individuals handling the airtight chamber, the piece of fabric-based currency 13 is hermetically sealed within the treatment chamber 2 by closing the chamber entrance/exit 3 (Step C). The hermetic seal prevents the individual from direct UV lamp 4 exposure, but more importantly, from any ozone exposure as O₃ is toxic for individuals.

The overall process continues by exposing the piece of fabric-based currency 13 to at least one quantity of UV radiation for a first specified period of time by activating the UV lamp 4 (Step D), which allows the present invention to target any pathogens located on the piece of fabric-based currency 13, also seen in FIG. 3. The at least one quantity of UV radiation significantly damages or destroys suspected pathogens. The first specified period of time depends on the type of pathogen or pathogens being targeted, the size of the UV lamp 4, and the distance between the UV lamp 4 and the fabric-based currency 13 within the treatment chamber 2. In order to target pathogens more integrated throughout the piece of fabric-based currency 13, the piece of fabric-based currency 13 is then exposed to a quantity of ozone for a second specified period of time by pumping the quantity of ozone into the treatment chamber 2 with the ozone dispensing system 5 (Step E). The quantity of ozone serves as an oxidizer which further damages or destroys microbes integrated within the piece of fabric-based currency 13 not reached by UV radiation. In the preferred embodiment of the present invention, the second specified period of time is between 30 to 60 seconds for sufficient sanitization. Moreover, the quantity of ozone is between 4 parts per million to 8 parts per million, thereby effectively targeting pathogens integrated within the piece of fabric-based currency 13 and while simultaneously preserving the structural integrity of the piece of fabric-based currency 13. After, the piece of fabric-based currency 13 is sufficiently sanitized, the chamber entrance/exit 3 is opened, and the piece of fabric-based currency 13 is removed from the treatment chamber 2 (Step F). Furthermore, in the preferred embodiment, Step B through Step F are sequentially executed as a series of steps, which allows the present invention to target pathogens from the outside and the inside. It is understood that for alternate embodiments of the present invention, the airtight container 1 may further include a storage compartment for the piece of fabric-based currency 13. The piece of fabric-based currency 13 is transferred from the treatment chamber 2 into the storage compartment after Step F for a desired period of time until the piece of fabric-based currency 13 is safe to be touched by the skin of an individual after ozone exposure during Step E. The storage compartment may also serve simply for the storage and organization of sanitized pieces of fabric-based currency 13.

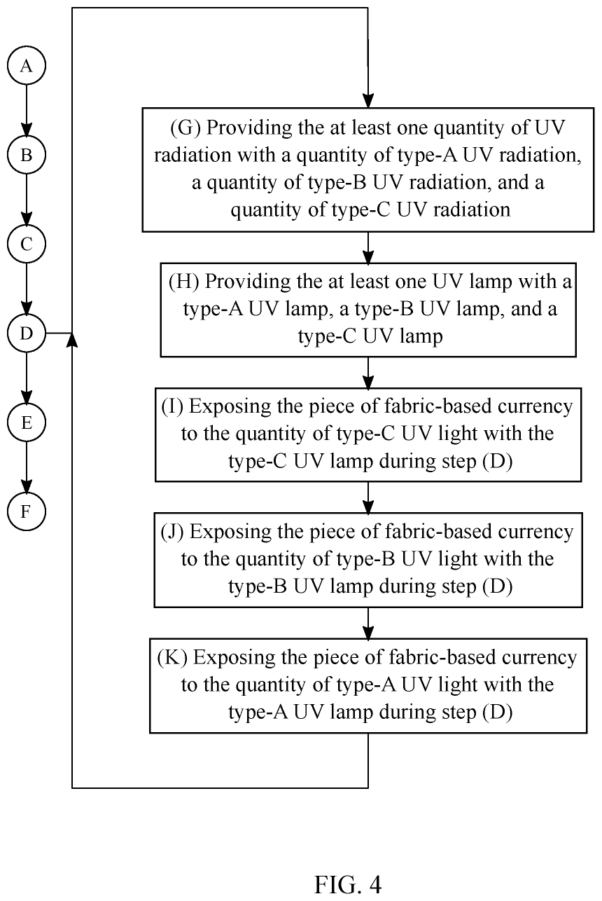

In some embodiments, the present invention allows for different types of UV radiation to be used to sanitize the piece of fabric-based currency. This, the at least one quantity of UV radiation includes a quantity of type-A UV radiation, a quantity of type-B UV radiation, and a quantity of type-C UV radiation (Step G), seen in FIG. 4. The quantity of type-A UV radiation, the quantity of type-B UV radiation, and the quantity of type-C UV radiation thoroughly target surface-level pathogens as different microbes respond to different types of radiation. The quantity of type-C UV radiation is between 200 nanometers to 280 nanometers. The quantity of type-B UV radiation is between 280 nanometers to 320 nanometers. The quantity of type-A UV radiation is between 315 nanometers to 400 nanometers. Moreover, in order to apply each type of radiation with the piece of fabric-based currency 13, the at least one UV lamp 4 includes a type-A UV lamp, a type-B UV lamp, and a type-C UV lamp (Step H). The piece of fabric-based currency 13 is initially exposed to the quantity of type-C UV radiation with the type-C UV lamp during Step D (Step I) as the quantity of type-C UV radiation has the strongest germicidal effect. The piece of fabric-based currency 13 is then exposed to the quantity of type-B UV radiation with the type-B UV lamp during Step D (Step J). The piece of fabric-based currency 13 is then exposed to the quantity of type-A UV lamp with the type-A UV lamp during Step D (Step K) for any remaining microbes missed by the exposure to the quantity of type-C UV radiation and the quantity of type-B UV radiation. Furthermore, Step I through Step K are sequentially executed as a series of steps for various microbes to be thoroughly targeted.

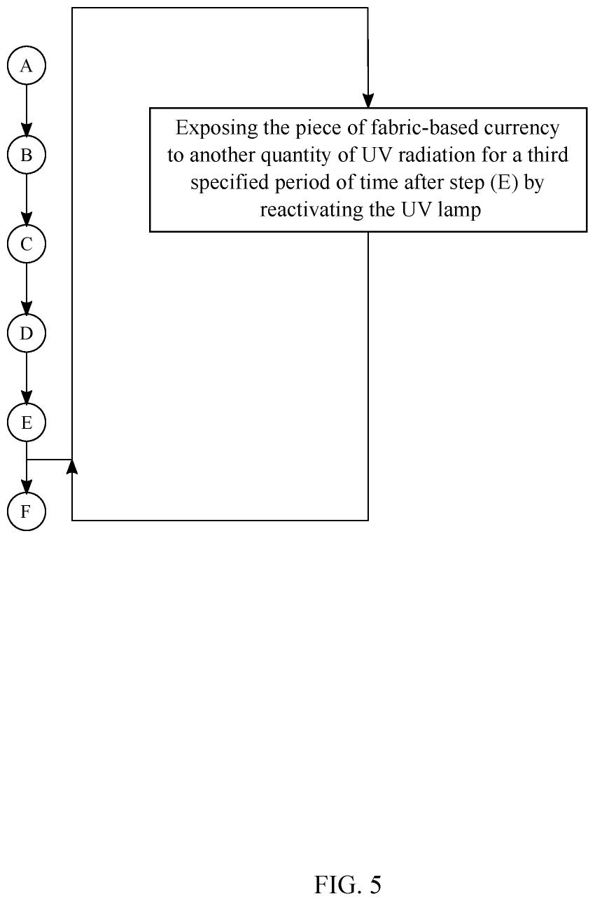

In order for the piece of fabric-based currency 13 to be safe to handle immediately upon removal from the treatment chamber 2, the piece of fabric-based currency 13 is exposed to another quantity of UV radiation for a third specified period of time after Step E by reactivating the UV lamp 4, as seen in FIG. 5. This additional quantity of UV radiation converts O₃ into O₂ for safe and immediate use by individuals during the third specified period of time. More specifically, this additional quantity of UV radiation renders O₃ molecules inert and the piece of fabric-based currency 13 safe to be touched by the skin of individuals. Furthermore, this additional quantity of UV radiation ensures pathogens remaining on the piece of fabric-based currency 13 are rendered inert or damaged. The third specified period of time depends on the type of pathogen or pathogens being targeted, the size of the UV lamp 4, and the distance between the UV lamp 4 and the fabric-based currency 13 within the treatment chamber 2.

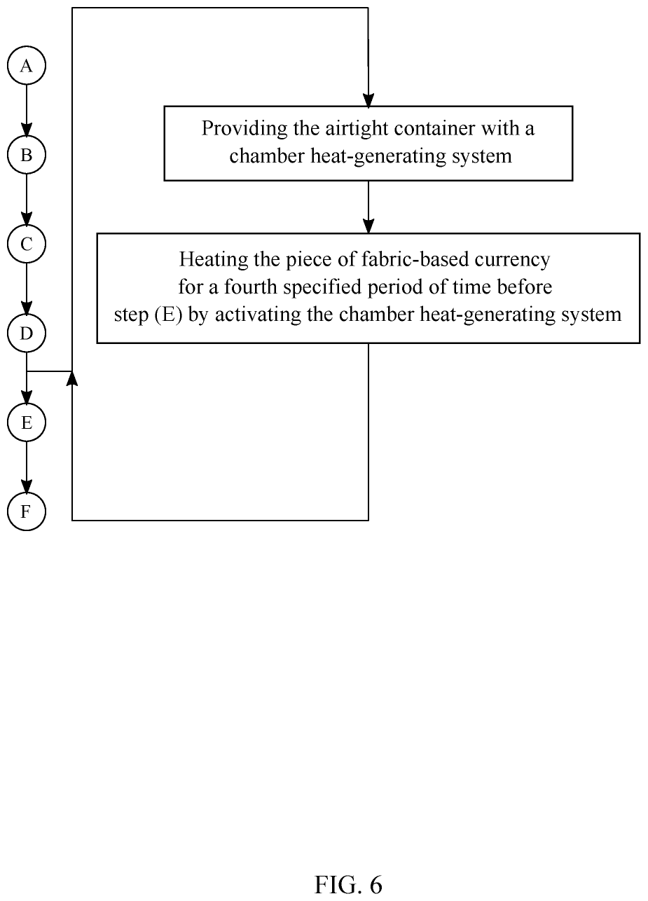

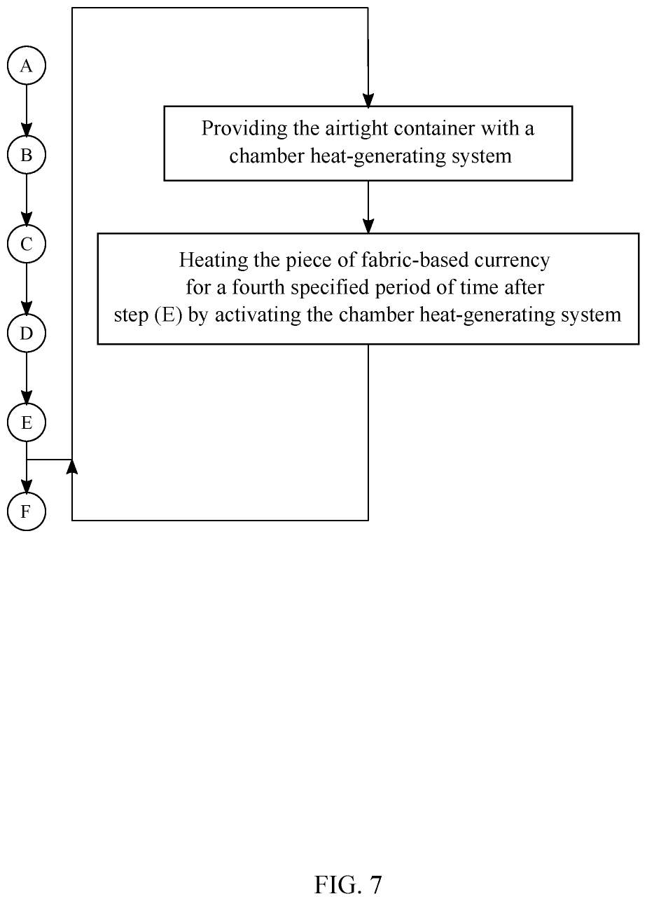

In order to further eliminate or significantly reduce the pathogens on the piece of fabric-based currency 13, the airtight container 1 further includes a chamber heat-generating system 14, as seen in FIG. 6. The chamber heat-generating system 14 serves as an additional precaution to sanitize the piece of fabric-based currency 13. The chamber heat-generating system 14 may further include an insulating layer that lines the treatment chamber 2 for efficient and effective heating, as well as a variety of heating elements within the treatment chamber 2. The chamber heat-generating system 14 exposes the piece of fabric-based currency 13 to high levels of thermal radiation in order to penetrate any solid matter or debris on the piece of fabric-based currency 13. Solid matter or debris are targeted on the piece of fabric-based currency 13 as the piece of fabric-based currency 13 is heated for a fourth specified period of time before Step E by activating the chamber heat-generating system 14. The amount of time heat is applied during the fourth specified period of time depends on how dirty the piece of fabric-based currency 13 is. The piece of fabric-based currency 13 is preferably heated to a temperature between 250 degrees Fahrenheit to 325 degrees Fahrenheit in order to significantly reduce pathogenic lifecycles. In another embodiment of the present invention, the piece of fabric-based currency 13 is heated for a fourth specified period of time after Step E by activating the chamber heat-generating system 14 in order to target solid matter or debris, seen in FIG. 7. Similarly, the fourth specified period of time depends on how dirty the piece of fabric-based currency 13 is, and the piece of fabric-based currency 13 is heated to a temperature between 250 degrees Fahrenheit to 325 degrees Fahrenheit. The piece of fabric-based currency 13 may be heated for the fourth specified period of time before Step E, after Step E, or both before Step E and after Step E depending on the degree of dirtiness of the piece of fabric-based currency 13.

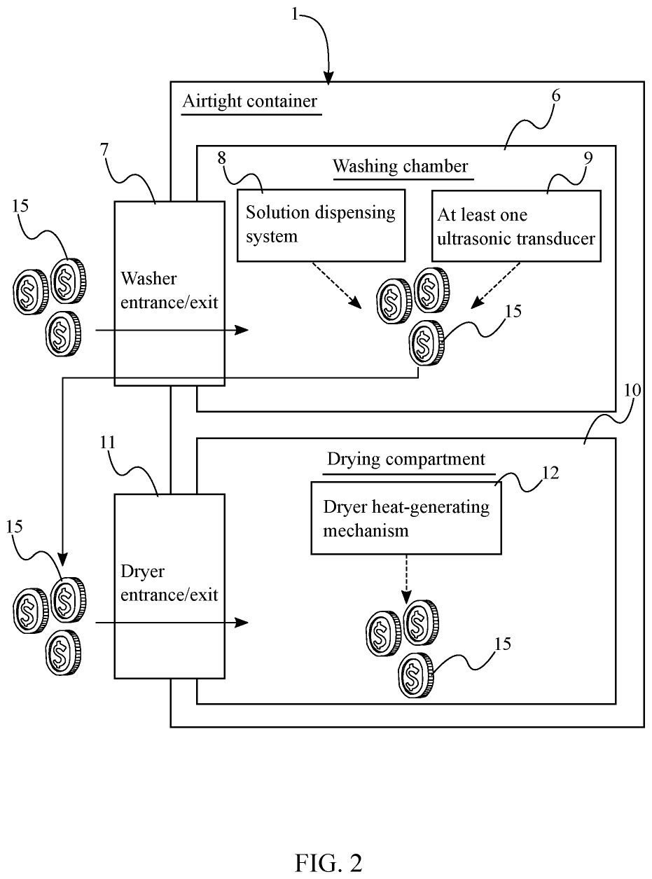

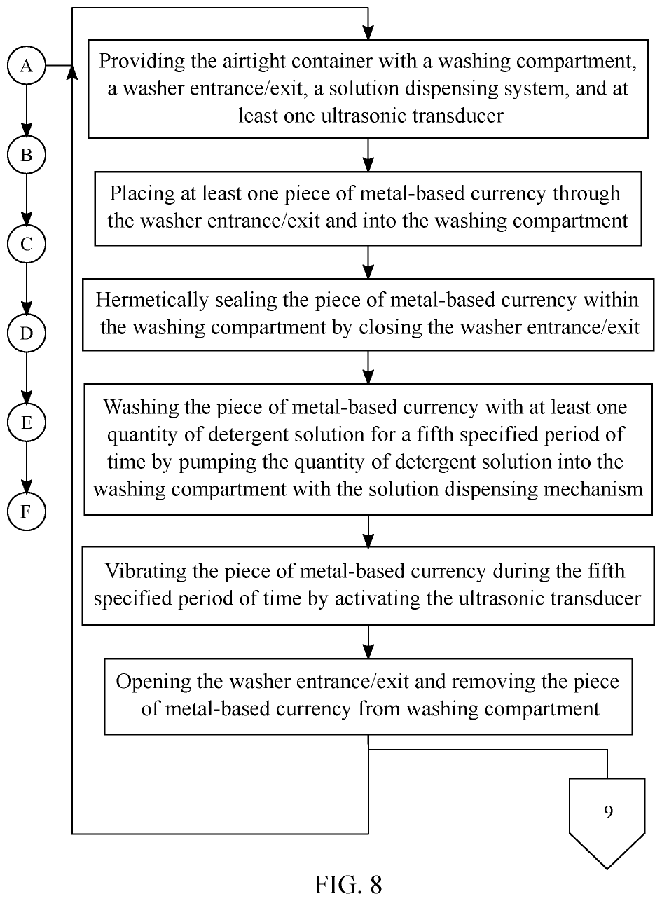

Further embodiments of the present invention may also sanitize metal-based currency 15 such as coins. In order to sanitize the metal-based currency 15 with the airtight container 1, the airtight container 1 includes a washing compartment 6, a washer entrance/exit 7, a solution dispensing mechanism 8, and at least one ultrasonic transducer 9, as seen in FIG. 2. The washing compartment 6 may simultaneously sanitize piece of metal-based currency 15 while the treatment chamber 2 treats at least one piece of fabric-based currency 13. The washer entrance/exit 7 provides access into and out of the washing compartment 6. In order to begin using the washing compartment, at least one piece of metal-based currency 15 is placed through the washer entrance/exit 7 and into the washing compartment 6, as seen in FIG. 8. Next, the piece of metal-based currency 15 is hermetically sealed within the washing compartment 6 by closing the washer entrance/exit 7. The piece of metal-based currency 15 is then washed with at least one quantity of detergent solution for a fifth specified period of time by pumping the quantity of detergent solution into the washing compartment 6 with the solution dispensing mechanism 8, thereby submerging and disinfecting the piece of metal-based currency 15. The quantity of detergent solution decontaminates the piece of metal-based currency 15 and breakdown cellular walls of pathogens during the fifth specified period of time. In order to destabilize pathogen cellular cohesion, the piece of metal-based currency 15 is simultaneously vibrated during the fifth specified period of time by activating the ultrasonic transducer 9. The applied amount of ultrasonic resonance may vary depending on the dirtiness of the piece of metal-based currency 15. The combination of the exposure to the quantity of detergent solution and the applied amount of ultrasonic resonance renders most active pathogens inert and sanitizes the piece of metal-based currency 15. In order to retrieve the sanitized piece of metal-based currency 15, the washer entrance/exit 7 is then opened, and the piece of metal-based currency 15 is removed from the washing compartment 6.

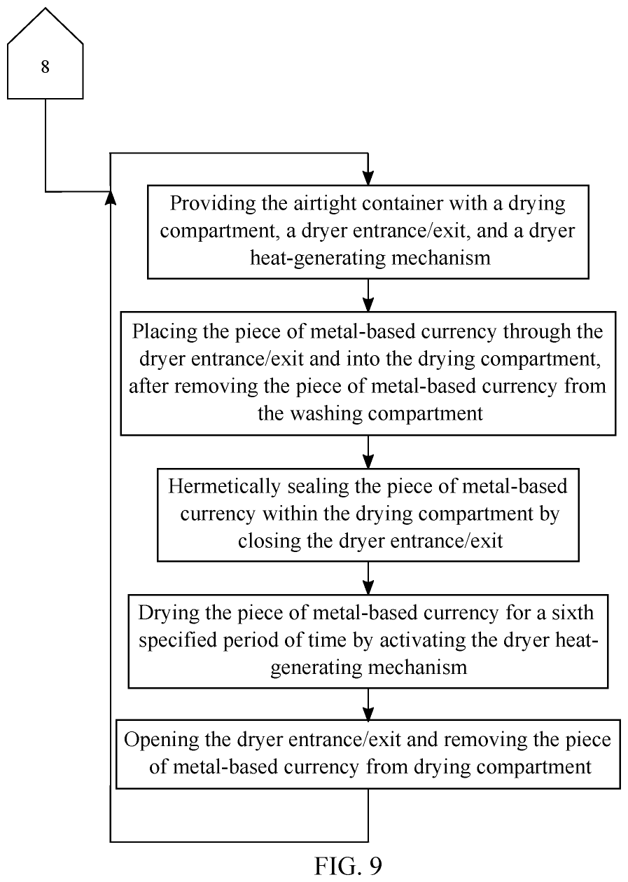

In these further embodiments of the present invention, the airtight container 1 further includes a drying compartment 10, a dryer entrance/exit 11, and a dryer heat-generating mechanism 12, also seen in FIG. 2. The drying compartment 10 heats the at least one piece of metal-based currency 15 in order to further sanitize the piece of metal-based currency 15 as well as facilitate the drying process of the piece of metal-based currency 15 so that the piece of metal-based currency 15 may be readily handled by an individual. The dryer entrance/exit 11 provides access into and out of the dryer compartment. The dryer heat-generating mechanism 12 emits heat within the drying compartment 10. In order to for the piece of metal-based currency 15 to be further sanitized and dried, the piece of metal-based currency 15 is placed through the dryer entrance/exit 11 and into the dryer compartment, after removing the piece of metal-based currency 15 from the washing compartment 6, seen in FIG. 9. The piece of metal-based currency 15 is hermetically sealed within the drying compartment 10 by closing the dryer entrance/exit 11, thereby containing the heat around the piece of metal-based currency 15 and within the drying compartment 10. In order to remove any quantity of detergent solution from the piece of metal-based currency 15, the piece of metal-based currency 15 is heated for a sixth specified period of time by activating the dryer heat-generating mechanism 12. The piece of metal-based currency 15 is thoroughly sanitized and ready to be handled by an individual after the sixth specified period of time. The amount of heat that is applied during the sixth specified period of time depends on how dirty the piece of metal-based currency 15 is and how wet the piece of metal-based currency 15 is. In order to retrieve the sanitized and dried piece of metal-based currency 15, the dryer entrance/exit 11 is opened, and the piece of metal-based currency 15 is removed from the drying compartment 10.

Although the invention has been explained in relation to its preferred embodiment, it is to be understood that many other possible modifications and variations can be made without departing from the spirit and scope of the invention as hereinafter claimed.

What is claimed is:

1. A method of sanitizing a fabric-based currency, the method comprising the steps:
(A) providing an airtight container, wherein the airtight container includes a treatment chamber, a chamber entrance/exit, at least one ultraviolet (UV) lamp, and an ozone dispensing system;
(B) placing at least one piece of fabric-based currency through the chamber entrance/exit and into the treatment chamber;
(C) hermetically sealing the piece of fabric-based currency within the treatment chamber by closing the chamber entrance/exit;
(D) exposing the piece of fabric-based currency to at least one quantity of UV radiation for a first specified period of time by activating the UV lamp;
(E) exposing the piece of fabric-based currency to a quantity of ozone for a second specified period of time by pumping the quantity of ozone into the treatment chamber with the ozone dispensing system; and,
(F) opening the chamber entrance/exit and removing the piece of fabric-based currency from treatment chamber.

2. The method as claimed in claim 1, wherein steps (B) through (F) are sequentially executed as a series of steps.

3. The method as claimed in claim 1, wherein the piece of fabric-based currency is made of a quantity of cotton and a quantity of linen, and wherein the quantity of cotton is 75 percent by weight of the piece of fabric-based currency, and wherein the quantity of linen is 25 percent by weight of the piece of fabric-based currency.

4. The method as claimed in claim 1 comprising the steps:
(G) providing the at least one quantity of UV radiation with a quantity of type-A UV radiation, a quantity of type-B UV radiation, and a quantity of type-C UV radiation;
(H) providing the at least one UV lamp with a type-A UV lamp, a type-B UV lamp, and a type-C UV lamp;
(I) exposing the piece of fabric-based currency to the quantity of type-C UV radiation with the type-C UV lamp during step (D);
(J) exposing the piece of fabric-based currency to the quantity of type-B UV radiation with the type-B UV lamp during step (D); and,
(K) exposing the piece of fabric-based currency to the quantity of type-A UV lamp with the type-A UV lamp during step (D).

5. The method as claimed in claim 4, wherein steps (I) through (K) are sequentially executed as a series of steps.

6. The method as claimed in claim 1, wherein the second specified period of time is between 30 seconds to 60 seconds.

7. The method as claimed in claim 1, wherein the quantity of ozone is between 4 parts per million to 8 parts per million.

8. The method as claimed in claim 1 comprising the steps:
exposing the piece of fabric-based currency to another quantity of UV radiation for a third specified period of time after step (E) by reactivating the UV lamp.

9. The method as claimed in claim 1 comprising the steps:
providing the airtight container with a chamber heat-generating system; and,
heating the piece of fabric-based currency for a fourth specified period of time before step (E) by activating the chamber heat-generating system.

10. The method as claimed in claim 9, wherein the piece of fabric-based currency is heated to a temperature between 250 degrees Fahrenheit to 325 degrees Fahrenheit.

11. The method as claimed in claim 1 comprising the steps:
providing the airtight container with a chamber heat-generating system; and,
heating the piece of fabric-based currency for a fourth specified period of time after step (E) by activating the chamber heat-generating system.

12. The method as claimed in claim 11, wherein the piece of fabric-based currency is heated to a temperature between 250 degrees Fahrenheit to 325 degrees Fahrenheit.

13. The method of sanitizing a fabric-based currency, the method as claimed in claim 1 comprising the steps:
providing the airtight container with a washing compartment, a washer entrance/exit, a solution dispensing mechanism, and at least one ultrasonic transducer;
placing at least one piece of metal-based currency through the washer entrance/exit and into the washing compartment;
hermetically sealing the piece of metal-based currency within the washing compartment by closing the washer entrance/exit;
washing the piece of metal-based currency with at least one quantity of detergent solution for a fifth specified period of time by pumping the quantity of detergent solution into the washing compartment with the solution dispensing mechanism;
vibrating the piece of metal-based currency during the fifth specified period of time by activating the ultrasonic transducer; and
opening the washer entrance/exit and removing the piece of metal-based currency from washing compartment.

14. The method of sanitizing a fabric-based currency, the method as claimed in claim 13 comprising the steps:
providing the airtight container with a drying compartment, a dryer entrance/exit, and a dryer heat-generating mechanism;
placing the piece of metal-based currency through the dryer entrance/exit and into the drying compartment, after removing the piece of metal-based currency from the washing compartment;
hermetically sealing the piece of metal-based currency within the drying compartment by closing the dryer entrance/exit;
drying the piece of metal-based currency for a sixth specified period of time by activating the dryer heat-generating mechanism; and
opening the dryer entrance/exit and removing the piece of metal-based currency from drying compartment.

15. A method of sanitizing a fabric-based currency, the method comprising the steps:
(A) providing an airtight container, wherein the airtight container includes a treatment chamber, a chamber entrance/exit, at least one ultraviolet (UV) lamp, and an ozone dispensing system;
(B) placing at least one piece of fabric-based currency through the chamber entrance/exit and into the treatment chamber, wherein the piece of fabric-based currency is made of a quantity of cotton and a quantity of linen, and wherein the quantity of cotton is 75 percent by weight of the piece of fabric-based currency, and wherein the quantity of linen is 25 percent by weight of the piece of fabric-based currency;
(C) hermetically sealing the piece of fabric-based currency within the treatment chamber by closing the chamber entrance/exit;
(D) exposing the piece of fabric-based currency to at least one quantity of UV radiation for a first specified period of time by activating the UV lamp;
(E) exposing the piece of fabric-based currency to a quantity of ozone for a second specified period of time by pumping the quantity of ozone into the treatment chamber with the ozone dispensing system, wherein the second specified period of time is between 30 seconds to 60 seconds, and wherein the quantity of ozone is between 4 parts per million to 8 parts per million;
(F) opening the chamber entrance/exit and removing the piece of fabric-based currency from treatment chamber; and
sequentially executing steps (B) through (F) are as a series of steps.

16. The method as claimed in claim 15 comprising the steps:
(G) providing the at least one quantity of UV radiation with a quantity of type-A UV radiation, a quantity of type-B UV radiation, and a quantity of type-C UV radiation;
(H) providing the at least one UV lamp with a type-A UV lamp, a type-B UV lamp, and a type-C UV lamp;
(I) exposing the piece of fabric-based currency to the quantity of type-C UV radiation with the type-C UV lamp during step (D);
(J) exposing the piece of fabric-based currency to the quantity of type-B UV radiation with the type-B UV lamp during step (D);
(K) exposing the piece of fabric-based currency to the quantity of type-A UV lamp with the type-A UV lamp during step (D); and
sequentially executing steps (I) through (K) as a series of steps.

17. The method as claimed in claim 15 comprising the steps:
exposing the piece of fabric-based currency to another quantity of UV radiation for a third specified period of time after step (E) by reactivating the UV lamp.

18. The method as claimed in claim 15 comprising the steps:
providing the airtight container with a chamber heat-generating system; and,
heating the piece of fabric-based currency for a fourth specified period of time before step (E) by activating the chamber heat-generating system, the piece of fabric-based currency is heated to a temperature between 250 degrees Fahrenheit to 325 degrees Fahrenheit.

19. The method as claimed in claim 15 comprising the steps:
providing the airtight container with a chamber heat-generating system; and,
heating the piece of fabric-based currency for a fourth specified period of time after step (E) by activating the chamber heat-generating system, the piece of fabric-based currency is heated to a temperature between 250 degrees Fahrenheit to 325 degrees Fahrenheit.

20. The method of sanitizing a fabric-based currency, the method as claimed in claim 15 comprising the steps:
providing the airtight container with a washing compartment, a washer entrance/exit, a solution dispensing mechanism, at least one ultrasonic transducer, a drying compartment, a dryer entrance/exit, and a dryer heat-generating mechanism;
placing at least one piece of metal-based currency through the washer entrance/exit and into the washing compartment;
hermetically sealing the piece of metal-based currency within the washing compartment by closing the washer entrance/exit;
washing the piece of metal-based currency with at least one quantity of detergent solution for a fifth specified period of time by pumping the quantity of detergent solution into the washing compartment with the solution dispensing mechanism;
vibrating the piece of metal-based currency during the fifth specified period of time by activating the ultrasonic transducer;
opening the washer entrance/exit and removing the piece of metal-based currency from washing compartment;
placing the piece of metal-based currency through the dryer entrance/exit and into the drying compartment, after removing the piece of metal-based currency from the washing compartment;
hermetically sealing the piece of metal-based currency within the drying compartment by closing the dryer entrance/exit;
drying the piece of metal-based currency for a sixth specified period of time by activating the dryer heat-generating mechanism; and
opening the dryer entrance/exit and removing the piece of metal-based currency from drying compartment.
